SEQUENCE LISTING (16 sequences)

SEQ ID NO: 1 - protein, 5 aa, Human
YGGFM

SEQ ID NO: 2 - protein, 5 aa, Human
YGGFL

SEQ ID NO: 3 - protein, 5 aa, Artificial
  Synthetic
YGGFM

SEQ ID NO: 4 - protein, 5 aa, Artificial
  Synthetic
YGGFL

SEQ ID NO: 5 - protein, 10 aa, Human
  PEPTIDE (1)..(10): Pro (residue 1) is 5-oxo-Pro. Gly (residue 10) is Gly-NH2.
PHWSYGLRPG

SEQ ID NO: 6 - protein, 9 aa, Artificial
  Synthetic
PHWSYLLRP

SEQ ID NO: 7 - protein, 10 aa, Artificial
  Synthetic
PHWSYALRPG

SEQ ID NO: 8 - protein, 9 aa, Artificial
  Synthetic
PHWSYSLRP

SEQ ID NO: 9 - protein, 9 aa, Artificial
  Synthetic
PHWSYHLRP

SEQ ID NO: 10 - protein, 10 aa, Artificial
  Synthetic
PHWSYKLRPG

SEQ ID NO: 11 - protein, 10 aa, Artificial
  Synthetic
PHWSYXLRPX

SEQ ID NO: 12 - protein, 9 aa, Artificial
  Synthetic
PHWSYLLRP

SEQ ID NO: 13 - protein, 10 aa, Artificial
  Synthetic
PHWSYKLRPG

SEQ ID NO: 14 - protein, 10 aa, Artificial
  Synthetic
PHWSYKLRPG

SEQ ID NO: 15 - protein, 8 aa, Artificial
  Synthetic
FCFWKTCT

SEQ ID NO: 16 - protein, 8 aa, Artificial
  Synthetic
FCFWKTCT

(19) United States
(12) Patent Application Publication
(10) Pub. No.: US 2005/0004000 A1
(43) Pub. Date: Jan. 6, 2005

(54) Oral absorbed drugs

(76) Inventors: Yoram Shechter, Rehovot (IL); Matityahu Fridkin, Rehovot (IL); Iris Lavon, Varburg (IL); David Brodie, East Windsor, NJ (US); Nurit Eyal, Ness Ziona (IL); Stanley Fass, Jerusalem (IL); Matityahu Fridkin, Rehovot (IL)

Correspondence Address: BROWDY AND NEIMARK, P.L.L.C.;624 NINTH STREET, NW, SUITE 300, WASHINGTON, DC, 20001-5303, US

(21) Appl. No.: 10/473,721

(22) PCT Filed: Mar. 26, 2002
(86) PCT No.: PCT/IL02/00252

(30) Foreign Application Priority Data
Apr. 1, 2001 (IL) 142353
Nov. 7, 2001 (IL) 146383

(51) Int. Cl. A61K 38/00; A61K 31/66; C07F 9/02

(52) U.S. Cl. 514/2; 514/114; 530/350; 558/209

(57) ABSTRACT

Orally nonabsorbed or poorly absorbed drugs may be converted to orally absorbed prodrug derivatives by derivatization of free functional groups selected from amino, hydroxyl, mercapto, phosphate and/or carboxyl with groups sensitive to mild basic conditions such as 9-fluorenylmethoxycarbonyl (Fmoc), 2-sulfo-9-fluorenylmethoxycarbonyl (Fms), and fluorenylmethyl (Fm).

### FIELD OF THE INVENTION

The present invention relates to novel orally deliverable prodrugs derived from orally nonabsorbed or poorly absorbed drugs, said prodrugs bearing functional groups sensitive to mild basic conditions such as 9-fluorenylmethoxycarbonyl (Fmoc), 2-sulfo-9-fluorenylmethoxycarbonyl (Fms), and fluorenylmethyl (Fm) groups, and to pharmaceutical compositions comprising them.

### BACKGROUND OF THE INVENTION

Certain therapeutical drugs used in human therapy and/or in veterinary are not absorbed or poorly absorbed orally and must be administered by other routes e.g. by injection, in order to reach the blood circulation.

Oral absorption of drugs is a highly desirable goal in the treatment of human diseases, particularly in prolonged therapeutical treatments. Major efforts are being made to convert orally non-absorbed or poorly absorbed drugs into orally absorbed drugs by encapsulation or by chemical modification. Structural alteration of drugs may result in an increase of the oral absorption, and, eventually, in biostability of the drugs.

International PCT Publication No. WO 98/05361 of the present applicants describes a new conceptual approach for prolonging the half-life of drugs, particularly proteins such as insulin, in vivo, and suggests that this approach may represent alternative possibilities for drug administration, e.g. oral and transdermal, and for penetration of the drug through physiological barriers. According to WO 98/05361, a drug containing a group selected from free amino, carboxyl, hydroxyl and/or mercapto is derivatized with a hydrophobic group such as 9-fluorenylmethoxycarbonyl (Fmoc) or 2-sulfo-9-fluorenylmethoxycarbonyl (Fms), chemical modifications which are reversible under physiological conditions. Moreover, such covalent modification renders the drugs, particularly proteins such as insulin, more stable towards enzymatic degradation, and increases the hydrophobicity index of the drug.

However, although Fmoc- and Fms-drug derivatives having increased resistance to proteolysis and increased lipophilicity may be candidates for use in orally active delivery systems, it is not obvious that polar hydrophlic molecules such as peptides, amino-sugars, amino acids and the like, that are not orally absorbed or are only poorly absorbed orally, will turn into orally absorbed species following enhancement of hydrophobicity. It has to be stressed that Fmoc- and Fms-modified drugs consist schematically of two domains: a polar domain and a hydrophobic domain, and the effect of each of these domains on oral delivery of the drug cannot be predicted, especially when the molecule has a high molecular weight.

### SUMMARY OF THE INVENTION

It is an object of the present invention to provide orally deliverable prodrugs derived from orally nonabsorbed or poorly absorbed drugs.

The present invention thus relates to an orally absorbed prodrug of the formula:
X—Y
wherein
Y is a moiety of an orally nonabsorbed or poorly absorbed drug bearing at least one functional group selected from free amino, hydroxyl, mercapto, phosphate and/or carboxyl, and
X is a radical selected from radicals of the formulas (i) to (iv). 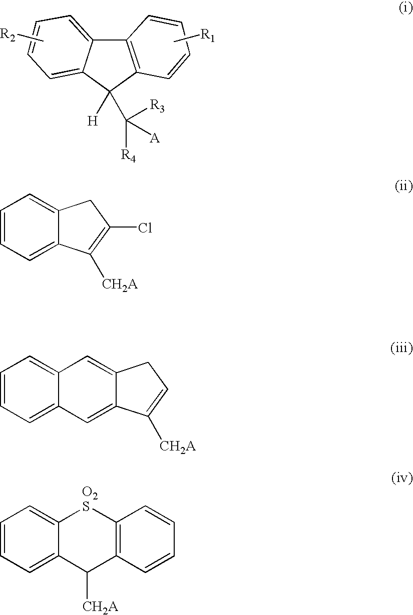
wherein R₁ and R₂, the same or different, are each hydrogen, alkyl, alkoxy, alkoxyalkyl, aryl, alkaryl, aralkyl, halogen, nitro, sulfo (SO₃H), amino, ammonium, carboxyl, PO₃H₂, or OPO₃H₂, R₃ and R₄, the same or different, are each hydrogen, alkyl or aryl; and A is a covalent bond when the radical is linked to a carboxyl, phosphate or mercapto group of the drug Y, or A is OCO— when the radical is linked to an amino or hydroxyl group of the drug Y, and pharmaceutically acceptable salts thereof.

In preferred embodiments of the invention, at least one functional group of the drug molecule Y is attached to at least one radical X wherein said radical X is the radical (i), wherein either R₁, R₂, R₃ and R₄ are hydrogen and A is OCO—, i.e. the 9-fluorenylmethoxycarbonyl radical (herein designated “Fmoc”) or R₁ is sulfo at position 2, R₂, R₃ and R₄ are hydrogen and A is OCO—, i.e. the 2-sulfo-9-fluorenylmethoxycarbonyl or 2-sulfo-Fmoc radical (herein designated “Fms”).

Several types of orally nonabsorbed or poorly absorbed drugs can be derivatized according to the invention thus obtaining orally absorbed prodrugs that are administered orally and are hydrolyzed to the original active drug molecule under physiological conditions.

The invention also relates to a method for converting an orally nonabsorbed or poorly absorbed drug to an orally absorbed drug suitable for oral delivery, which comprises attaching to at least one free amino, hydroxy, mercapto, phosphate and/or carboxyl group of said orally nonabsorbed or poorly absorbed drug at least one radical selected from the group consisting of the radicals of the formulas (i) to (iv) as described above.

The invention further relates to pharmaceutical compositions for oral administration comprising a prodrug X—Y of the invention or a pharmaceutically acceptable salt thereof, and a pharmaceutically acceptable carrier.

In another aspect, the invention provides a method for oral treatment of a disease or disorder that can be treated with an orally nonabsorbed or poorly absorbed drug Y, which comprises administering to an individual in need thereof a suitable amount of a prodrug X—Y of the invention or a pharmaceutically acceptable salt thereof.

### BRIEF DESCRIPTION OF THE FIGURES

FIG. 1 shows that Fms-doxorubicin (Fms-Dox) is absorbed upon oral administration as demonstrated by fluorescence units in urine of rats. Rats received native doxorubicin (Dox) or Fms-Dox (10 μM/rat, namely 10 mg/kg of Dox or 15 mg/kg of Fms-Dox) by oral administration. Urine was collected 1.5, 2.5, 4 and 6 hours after administration and the fluorescence units were detected by fluorometer.

FIG. 2 shows that Fms-Dox kills cancer cells in vitro. Cells were treated with a range of concentrations of Dox and Fms-Dox and incubated for 96 hours before being assayed by crystal violet staining.

FIG. 3 shows that Fmoc-Met-enkephalin has analgesic activity. Thirty minutes after subcutaneous administration of the appropriate treatment, acetylcholine (5.5 mg/kg) was injected intraperitoneally (10 mice per group). The mice were then placed in large plastic boxes and observed for the occurrence of a single abdominal constriction.

FIG. 4 shows that Fmoc-Met-enkephalin is orally absorbed. Thirty and 120 minutes after administration of the appropriate treatment, acetylcholine (5.5 mg/kg) was injected intraperitoneally (10 mice per group). The mice were then placed in large plastic boxes and observed for the occurrence of a single abdominal constriction.

### DETAILED DESCRIPTION OF THE INVENTION

According to the present invention, orally absorbed prodrug derivatives obtained from orally non-absorbed or poorly absorbed drugs may be prepared by reaction of the parent drug molecule with a suitable reagent comprising a radical (i) to (iv) as defined herein.

In the radical (i), R₁ and R₂, the same or different are each hydrogen, alkyl, alkoxy, alkoxyalkyl, aryl, alkaryl, aralkyl, halogen, nitro, sulfo (SO₃H), amino, ammonium, carboxyl, PO₃H₂, or OPO₃H₂, R₃ and R₄, the same or different, are each hydrogen, alkyl or aryl.

The term “alkyl” as used herein in the terms “alkyl”, “alkoxy”, “alkoxyalkyl”, “alkaryl” and “aralkyl” denotes an alkyl radical 1-8, preferably 1-4carbon atoms, e.g. methyl, ethyl, propyl, isopropyl and butyl, and the term “aryl” as used herein in the terms “aryl”, “alkaryl” and “aralkyl” denotes a carbocyclic aryl radical of 6-10 carbon atoms, e.g. phenyl and naphthyl. The term “halogen” includes bromo, fluoro, chloro and iodo.

In one preferred embodiment of the invention, the functional group is the radical (i), wherein R₁, R₂, R₃ and R₄ are hydrogen and A is OCO—, i.e. the 9-fluorenylmethoxycarbonyl (Fmoc). The Fmoc group is widely used in peptide synthesis for the temporary and reversible protection of amino groups and is particularly suitable for peptide synthesis due to favorable synthetic manipulation for its introduction and removal, and preferential stability as a prerequisite for peptide synthesis and convenient purification.

In another preferred embodiment of the invention, the functional group is the radical (i), wherein R₁ is sulfo at position 2, R₂, R₃ and R₄ are hydrogen and A is OCO—, i.e. the 2-sulfo-9-fluorenylmethoxycarbonyl or 2-sulfo-Fmoc (Fms) radical. Sulfonation of the Fmoc group concomitantly introduces hydrophobic and substantial polar properties.

In a further embodiment of the invention, the functional group is the radical (i), wherein R₁, R₂, R₃ and R₄ are hydrogen and A is a covalent bond, i.e. the 9-fluorenylmethyl (herein designated “Fm”) radical. The Fm group is applicable for reversible masking of carboxylic functions of amino acids. The resulting 9-fluorenylmethyl esters (Fm-esters) generate the parent free carboxylic functions following a β-elimination reaction pathway upon mild basic treatment, i.e., under physiological conditions, and thus can be employed for reversible masking of carboxylic functions of drugs.

The halogenated Fmoc radicals (i) wherein at least one of R₁ and R₂ is halogen in the 2 or 7 position, preferably Cl or Br, the 2-chloro-1-indenylmethoxy-carbonyl (Climoc) radical (ii), the 1-benzo[f]indenylmethoxycarbonyl urethane (Bimoc) radical (iii), the urethane sulfone radical (iv) and corresponding radicals (i) to (iv) wherein A is a covalent bond, can be used similarly to Fmoc, Fms and Fm for substitution of free amino, carboxyl, hydroxyl, phosphate and mercapto functions of drugs, thus providing a wide range of sensitivity toward removal of such groups under basic, e.g. physiological, conditions.

In fact, the above radicals (i) to (iv) belong to a general family of rare chemical entities that undergo hydrolysis at neutral or slightly alkaline pH and mild conditions, and can therefore be used for temporary reversible protection of α- and ε-amino groups, for example in peptide synthesis, and can be removed from the amino function by a β-elimination reaction, under mild basic conditions.

According to the invention, a radical (i) to (iv), preferably Fmoc and Fms, covalently linked to amino and/or hydroxyl moieties, or Fm covalently linked to carboxyl, phosphate and/or mercapto moieties may, but does not necessarily, undergo hydrolysis (via β-elimination) back to the free amino, hydroxy, mercapto, phosphate or carboxyl functions, under physiological conditions in the body fluid, namely at pH 7.4 and 37° C.

In one preferred embodiment of the invention, at least one free amino and/or carboxyl group, and optionally at least one free hydroxyl group, of the parent drug molecule, are substituted by at least one radical of the formula (i) above, more preferably, one or more amino groups are substituted by Fmoc (herein Fmoc-drug) or by Fms (herein Fms-drug).

According to further embodiments of the invention, at least one carboxyl group of the parent drug molecule is substituted by Fm (herein Fm-drug); or at least one amino group is substituted by Fmoc and at least one carboxyl group is substituted by Fm (herein N-Fmoc, C-Fm-drug); or at least one carboxyl group is substituted by Fm and at least one hydroxyl group is substituted by Fmoc (herein C-Fm, O-Fmoc-drug); or at least one amino and at least one hydroxyl groups are substituted by Fmoc and at least one carboxyl group is substituted by Fm (herein N,O-Fmoc, C-Fm-drug).

For the preparation of the compounds of the invention several reagents are available such as N-(9-fluorenylmethoxycarbonyloxy)succinimide (Fmoc-OSu) and N-(2-sulfo-9-fluorenylmethoxycarbonyloxy) succinimide (Fms-OSu), that are very specific for amino functions, 9-fluorenylmethoxycarbonyl chloride (Fmoc-Cl), that reacts with, and covalently attaches to, amino and hydroxyl groups; 9-chloromethylfluorene (Fm-Cl), that reacts with mercapto radicals to yield S-Fm derivatives; and 9-fluorenylmethanol (Fm-OH), that reacts with, and esterifies, carboxylic and phosphate functions.

In one preferred embodiment of the invention, the orally nonabsorbed or poorly absorbed drug is a drug containing an amino-sugar moiety that can be derivatized according to the invention by substitution of at least one of the amino groups by Fmoc or Fms.

According to one embodiment, the orally nonabsorbed or poorly absorbed drug containing an amino-sugar moiety is an anthracycline antibiotic such as daunorubicin, sued in the treatment of acute leukemia, or doxorubicin (previously known as adriamycin), used as antineoplastic agent in chemotherapy of different types of cancer. Both drugs are presently administered intravenously. They can be converted according to the invention, for example, to orally absorbed Fmoc- or Fms-daunorubicin and Fmoc- or Fms-doxorubicin.

According to another embodiment, the orally nonabsorbed or poorly absorbed drug containing an amino-sugar moiety is an antibacterial aminoglycoside antibiotic such as streptomycin, tobramycin and gentamicin, which are presently administered intramuscularly on intravenously, and can be converted to orally absorbed Fmoc and Fms derivatives, for example, to (Fms)₃-gentamicin.

According to a further embodiment, the orally nonabsorbed or poorly absorbed drug containing an amino-sugar moiety is an antifungal polyene antibiotic such as amphotericin B, which is presently administered intravenously and can be converted to orally absorbed Fms-amphotericin B.

In another embodiment of the present invention, the orally nonabsorbed or poorly absorbed drug is a drug containing at least one carboxylic group that can be derivatized according to the invention by substitution of at least one of the carboxyl groups by Fm.

According to one embodiment, the orally nonabsorbed or poorly absorbed drug containing at least one carboxyl group is a beta-lactam antibiotic such as the semi-synthetic third generation cephalosporine ceftazimide and the broad spectrum semi-synthetic penicillins meropenem. These antibiotics are used only in hospitals, in particular for treatment of hospital acquired infections due to resistant organisms. They are presently administered intravenously 3-4 times a day and can be converted to corresponding orally absorbed Fm-derivatives according to the invention by derivatization of the free carboxyl groups with Fm-OH.

In another embodiment of the invention, the orally nonabsorbed or poorly absorbed drug is a peptide.

In one preferred embodiment of the invention, the orally nonabsorbed or poorly absorbed peptide is a peptide of the endorphin class. Endorphin is a generic name for a group of neuropeptides that are endogenous ligands of the opiate receptors and whose effects resemble those of opiates such as morphine and heroin because they bind to the same receptor in certain cells of the brain and induce, for example, analgesia. In one embodiment of the invention, the orally nonabsorbed or poorly absorbed drug is Met⁵-enkephalin or Leu⁵-enkephalin, two naturally occurring pentapeptides belonging to the endorphin class, of the sequences herein denoted as SEQ ID NOs: 1 and 2, respectively

| | |
|---|---|
| Tyr-Gly-Gly-Phe-Met | SEQ ID NO:1 |
| Tyr-Gly-Gly-Phe-Leu | SEQ ID NO:2 |

Met-enkephalin and Leu-enkephalin can be converted according to the invention to orally absorbed Fmoc and Fms derivatives. It should be noted that the Fmoc and Fms radicals are linked to the amino terminal group of the Tyr residue, but for reasons of convenience the compounds are herein denoted as Fmoc-Met-enkephalin, Fms-Met-enkephalin, Fmoc-Leu-enkephalin and Fms-Leu-enkephalin. Preparation of the Fmoc derivatives can be carried out by automatic peptide chain assembly on solid support and then cleaving the peptide from the resin without deprotecting its amino-terminal i.e. keeping the Fmoc moiety bound.

Fmoc-Met-enkephalin, Fms-Met-enkephalin, Fmoc-Leu-enkephalin and Fms-Leu-enkephalin have the sequences as denoted by SEQ ID NOs: 3 and 4, respectively:

| | |
|---|---|
| (X₁)Tyr-Gly-Gly-Phe-Met | SEQ ID NO: 3 |
| (X₁)Tyr-Gly-Gly-Phe-Leu | SEQ ID NO: 4 |

wherein X₁ is Fmoc or Fms.

In another preferred embodiment of the invention, the orally nonabsorbed or poorly absorbed peptide is a peptide hormone selected from gonadotropin releasing hormone (GnRH) or an analogue thereof, and octreotide.

Gonadotropin-releasing hormone (GnRH), also known as gonadotropin-releasing factor, luteinizing-hormone releasing factor or gonadorelin, is a decapeptide found in all mammals, of the sequence herein denoted as SEQ ID NO: 5 (the 5-oxo proline at the amino terminal is sometimes presented as pyroglutamic acid—pGlu):

SEQ ID NO: 5

5-oxo-Pro-His-Trp-Ser-Tyr-Gly-Leu-Arg-Pro-Gly-NH₂

GnRH is a neurohormone produced in the hypothalamic neurosecretory cells that controls release of the gonadotropins luteinizing hormone (LH) and follicle-stimulating hormone (FSH) from the anterior pituitary Divergent production of the two gonadotropins is controlled by the frequency of pulsatile GnRH secretion or administration and serum estradiol levels. GnRH is thus a key integrator between the neural and the endocrine system and plays a pivotal role in the regulation of the reproductive system. GnRH is presently administered subcutaneously or intravenously for diagnostic use, for example to test pituitary LH responsiveness, or in the treatment of female or male infertility.

Several synthetic analogs of GnRH are known in which the glycine residue at position 6 is replaced by a D-amino acid hence making the peptide less susceptible to proteolytic degradation. In addition, the glycine at position 10 of GnRH may be deleted or replaced by an ethylamide or aminocarbonylhydrazide group.

Leuprolide (6-D-Leu-9-(N-ethyl-L-prolinamide)-10-deglycynamide-GnRH) is a synthetic nonapeptide agonist analog of GnRH used in the treatment of endometriosis and uterine fibroids, central precocious puberty and prostate cancer. It is administered by subcutaneous or intramuscular injection. Leuprolide has the sequence denoted by SEQ ID NO: 6:

| | |
|---|---|
| 5-oxo-Pro-His-Trp-Ser-Tyr-D-Leu-Leu-Arg-Pro-NHCH₂CH₃ | SEQ ID NO: 6 |

Nafarelin {6-[3-(2-naphthalenyl)-D-alanine]GnRH or D-Nal⁶GnRH, wherein D-Nal is D-3-(2-naphthyl)-alanine)} is another synthetic peptide agonist analog of GnRH used in the form of a nasal preparation in the treatment of endometriosis and central precocious puberty. Nafarelin has the sequence denoted by SEQ ID NO: 7:

| | |
|---|---|
| 5-oxo-Pro-His-Trp-Ser-Tyr-D-Nal-Leu-Arg-Pro-Gly-NH₂ | SEQ ID NO: 7 |

Goserelin {6-[0-(1,1-dimethylethyl)-D-serine]-10-deglycynamide-GnRH 2-(aminocarbonyl) hydrazide} is a synthetic nonapeptide agonist analog of GnRH used in the treatment of endometriosis, dysfunctional uterine bleeding, and breast and prostate cancer. It is available as in the form of implantable cylinders that are placed subcutaneously in the upper abdominal area. Goserelin has the sequence denoted by SEQ ID NO: 8:

5-oxo-Pro-His-Trp-Ser-Tyr-D-Ser(t-Bu)-Leu-Arg-Pro-NHNHCONH₂

Histrelin {6-[1-(phenylmethyl)-D-histidine]-9-(N-ethyl-L-prolinamide)-10-deglycynamide-GnRH} is another synthetic nonapeptide agonist analog of GnRH used in the treatment of central precocious puberty. It is administered by subcutaneous or intramuscular injection. Histrelin has the sequence denoted by

| | |
|---|---|
| 5-oxo-Pro-His-Trp-Ser-Tyr-D-His(N^{t}-PhCH₂)-Leu-Arg-Pro-NHCH₂CH₃ | SEQ ID NO: 9: |

A new synthetic agonist analog of GnRH has been recently described, in which the glycine residue at position 6 was replaced by D-Lysine (Rahimipour, S., et Al. J. Med. Chem. 2001, 44, 3645-3652). This analog, herein D-Lys⁶GnRH, has the sequence denoted by SEQ ID NO: 10.

| | |
|---|---|
| 5-oxo-Pro-His-Trp-Ser-Tyr-D-Lys-Leu-Arg-Pro-Gly-NH₂ | SEQ ID NO: 10 |

Continuous administration of GnRH or of its superactive agonists results in downregulation of GnRH receptors and desensitization of the pituitary gonadotrophs, which bring about the suppression of gonadotropin secretion.

GnRH can stimulate pituitary function and is used to treat infertility caused by hypothalamic hypogonadotropic hypogonadism in both sexes. Synthetic GnRH analog agonists such as leuprolide, nafarelin, goserelin and histrelin induce hypogonadism when given continuously and are used, as mentioned above, for treatment of endometriosis, uterine fibroids, polycystic ovary syndrome, central precocious puberty and as antineoplastic (hormonal) drugs for the treatment of prostate or breast cancer.

GnRH and all known GnRH analogs currently used as drugs are administered by injection and there is interest in converting them to orally absorbed drugs. According to the present invention, both GnRH and GnRH analogs can be converted to orally absorbed drugs by substitution of a free hydroxyl or amino group by Fmoc or Fms. Thus, as a way of example, leuprolide was converted to FMS-leuprolide by substitution of the free hydroxyl group of the tyrosine at position 5 by Fms and D-Lys⁶GnRH was converted to Fms-D-Lys⁶-GnRH and Fmoc-D-Lys⁶-GnRH by substitution of the free amino group of the D-lysine at position 6 by Fms and Fmoc, respectively.

Thus, the present invention further contemplates Fmoc and Fms derivatives of GnRH and analogs thereof of the sequence denoted by SEQ ID NO:11:
R₅-5-oxo-Pro-His-Trp-Ser-Tyr-R₆-Leu-Arg-Pro-R₇
wherein R₅ is a Fmoc or Fms substitution at a free amino or hydroxyl group of an amino acid residue; R₆ is Gly or a D-amino acid residue selected from a natural or non-natural amino acid such as D-Leu, D-Lys, D-Nal [D-3-(2-naphthyl)-alanine], D-Ser(t-Bu) or D-His(N^{τ}-PhCH₂), and R₇ is Gly-NH₂, NHCH₂CH₃ or NHNHCONH₂.

The Fmoc or Fms substitution may be, for example, at the free hydroxyl group of Ser or Tyr or at the free amino group of Lys when R₆ is D-Lys.

In one preferred embodiment, the modified GnRH analog according to the invention is Fms-leuprolide, in which the free hydroxyl group of the tyrosine at position 5 is substituted by Fms, of the SEQ ID NO: 12:

5-oxo-Pro-His-Trp-Ser-(Fms)Tyr-D-Leu-Leu-Arg-Pro-NHCH₂CH₃

In another preferred embodiment, the modified GnRH analog according to the invention is Fmoc- or Fms-D-Lys⁶GnRH, in which the free amino group of the D-lysine at position 6 is substituted by Fmoc or Fms, of the SEQ ID NOs: 13 and 14, respectively:

| | |
|---|---|
| 5-oxo-Pro-His-Trp-Ser-Tyr-(Fmoc)D-Lys-Leu-Arg-Pro-Gly-NH₂ | SEQ ID NO: 13 |
| 5-oxo-Pro-His-Trp-Ser-Tyr-(Fms)D-Lys-Leu-Arg-Pro-Gly-NH₂ | SEQ ID NO: 14 |

In a further embodiment, the orally nonabsorbed or poorly absorbed hormone peptide is octreotide, a synthetic analogue of somatostatin having actions similar to somatostatin. Octreotide is a cyclic eight-amino acid peptide of the sequence denoted by SEQ ID NO: 15:

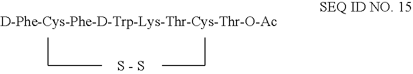

Octreotide inhibits growth hormone release and is used to suppress or inhibit certain symptoms associated with hormone-secreting tumors in patients with acromegaly, carcinoid tumors, and other syndromes and also for the acute control of bleeding from esophageal varices. Octreotide is administered subcutaneously and can be converted to an orally absorbed derivative by functionalizing with Fmoc or Fms the free amino group of the Lys residue at position 5 of the sequence as shown below (SEQ ID NO:16): 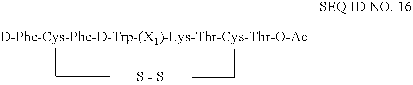
wherein X₁ is Fmoc or Fms.

In still a further embodiment, the orally nonabsorbed or poorly absorbed peptide is eptifibatide, a glycoprotein inhibitor of the antiplatelet family, which targets the platelet IIb/IIIa receptor complex. Eptifibatide belongs to a new class of antiplatelet agents and is beneficial in the treatment of acute coronary syndrome and percutaneous coronary angioplasty. Eptifibatide is administered parenterally and can be derivatized to an orally absorbed from according to the invention by substitution of the free amino group of the Lys residue with Fmoc or Fms.

In another aspect, the present invention relates to oral pharmaceutical compositions comprising the orally absorbed prodrug or a pharmaceutically acceptable salt thereof and a pharmaceutically acceptable carrier. These preparations can be made by conventional methods known to those skilled in the art, for example as described in “Remington's Pharmaceutical Science”, A. R. Gennaro, ed., 17th edition, 1985, Mack Publishing Company, Easton, Pa., USA.

In a further aspect, the invention relates to a method for treatment of a disease or disorder that can be treated with a drug that is not orally absorbed or is only poorly absorbed, which comprises administering to an individual in need thereof an orally absorbed derivative of said drug according to the invention.

In still another aspect, the invention relates to a method for oral delivery of an orally nonabsorbed or poorly absorbed drug, which comprises attaching to at least one free amino, hydroxy, mercapto, phosphate and/or carboxyl group of said drug a radical selected from radicals of the formulas (i) to (iv) herein.

The invention will now be illustrated by the following non-limiting Examples.

### EXAMPLES

### Materials and Methods

(a) Materials

(i) N-fluorenylmethoxycarbonyloxy)succinimide (Fmoc-OSu) was purchased from Novabiochem (Laüfelfingen, Switzerland).

(ii) N-(2-Sulfo-9-fluorenylmethoxycarbonyloxy) succinimide (Fms-OSu) was prepared essentially by the procedure of Merrifield and Bach, 1978, with slight modifications. Fmoc-OSu (337.4 mg, 1 mmole) was dissolved in 4 ml dichloromethane and the solution cooled to 0° C. A cold solution of ClSO₃H (60 μl, 0.9 mmole) in 2 ml dichloromethane was added with constant stirring and cooling (0° C.) over a period of 15 min. The yellow-turning solution was allowed to warm to room temperature and a white precipitate was formed within one hour. At two hours, cyclohexane (4 ml) was added to dissolve the unreacted Fmoc-OSu. The suspension was centrifuged and the solid material washed 4 times with 6 ml of 1:1 cyclohexane:dichloromethane. The white solid thus formed was dried under P₂O₅ in vacuo for 24 h and had the following characteristics: yield—290 mg (86%); mp 140-146° C.; TLC (1-butanol:acetic acid:water, 8:1:1) Rf 0.31, and mass spectrum (ES); m/z 416 (100% M−1). (Fms moieties, either free or covalently bound to proteins, absorb at the U.V range with Molars Extinction Coefficients ε280=21,200 and ε301=10,300.

(iii) Doxorubicin (Dox) was obtained from TEVA Pharmaceutical Industries Ltd., Petach-Tikva, Israel.

All other materials used in the examples were of analytical grade.

### Example 1

### Synthesis of Fms-doxorubicin (Fms-Dox) and Fms-daunorubicin

Doxorubicin (Dox)-HCl (5 mg) and Na₂CO₃ (2 mg) were dissolved in 0.5 ml double-distilled water (DDW). Fms-Osu (1.5 mg) was dissolved in 0.5 ml disoxane DDW (1:1) and added into the reaction mixture. The pH was adjusted to 8.5 with Na₂CO₃. Following one hour of stirring at room temperature, the remaining reagent was freshly dissolved (1:1 DDW:dioxane, 0.5 ml) and added. The reaction was monitored by analytical HPLC: C18 column, flow 0.8 ml/min. Fms-doxorubicin was purified by preparative HPLC on a C-18 column, employing a binary gradient formed from solution A: 0.1% TFA in H₂O and solution B: 0.1% TFA in 25% H₂O in acetonitrile. The gradient was t=0, 80% A; t=5, 80% A; t=65, 100% B; t=75, 100% B, at a flow of 8 ml/min. Retention time: 29.3 min. Mass spectrometry: m/z 862.

In an alternative procedure, doxorubicine (50 mg, MW 543, 1 equivalent) was dissolved in 2 ml of water to which 2 ml dioxane was added. Fms-OSu (43 mg, MW 417, 1.2 equivalent) were dissolved in 0.5 ml water and were added in two portions separated by one hour. The pH was adjusted to 8-8.5 with 1N Na₂CO₃ and the solution was stirred for 3 hours. The resulting mixture was analyzed by HPLC employing the binary system described above at a flow of 0.8 ml/min. Two peaks appeared at around 22 and 24 minutes corresponding to two isomers of Fms-Dox. The two were isolated by preparative HPLC utilizing the following binary system: t=0 to 15 min: 20% water/acetonitrile (25/75) in water; t=15-105 minutes 20-90% water/acetonitrile (25/75) in water. Separation was achieved at a flow of 12.5 ml/min using UV detection at 220 nm. The yield was quantitative based on starting material.

Fms-daunorubicin is prepared in the same way and tested for its activity as described below for Fms-doxorubicin.

### Example 2

### Fms-Dox is Absorbed Orally

In order to analyze whether Fms-Dox is orally absorbed, urine was collected following oral administration to mice (C57B16 mice, 7-9 week old) or to rates (100 g Wistar rats). The experiment was carried out with a single mice or rat per compound (per metabolic cage) and it was repeated 7 times with rats and twice with mice using the protocol below. The fluorescent feature of doxorubicin enables the observation of its presence in the urine following different modes of administration.

Rats were divided into 5 groups: (1) Control untreated rats; (2) rats treated with native Dox (10 μg/g) administered intraperitoneally (IP); (3) rats treated with Fms-Dox (15 μg/g) administered I.P.; (4) rats treated with native Dox (10 μg/g) administered orally; and (5) rats treated with Fms-Dox (15 μg/g) administered orally. Urine was collected several times after administration and the fluorescence emission was measured by a fluorometer. The urine collected from the untreated groups was used as a blank.

The experiments were repeated with mice. As expected, native Dox and Fms-Dox were absorbed in both species following I.P. administration. Moreover, it was demonstrated that Fms-Dox is absorbed orally in both species, unlike native doxorubicin when administered orally (not shown). Thus an orally nonabsorbed drug turned into an absorbed prodrug.

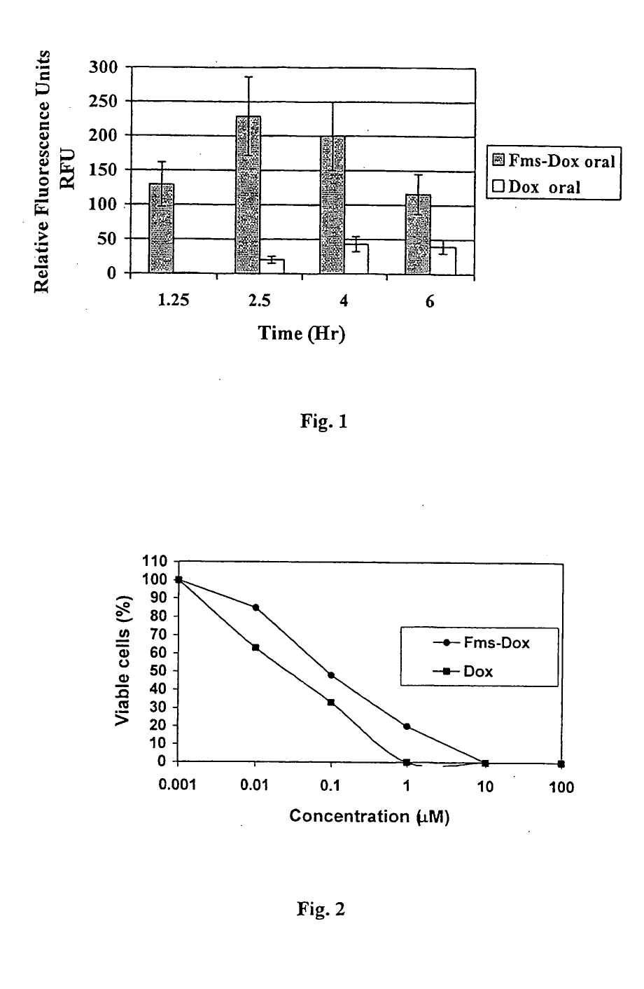

In another experiment, rats received either native Dox or Fms-Dox (10 μM/rat, namely 10 mg/Kg of Dox and 15 mg/Kg of Fms-Dox) by oral administration. Urine was collected at 1.25, 2.5, 4 and 6 hours after administration and fluorescence intensity was measured in a fluorometer (SPECTRAmax and GEMINI of Molecular Devices Sunnyvale Fluorometer, CA, USA). Since doxorubicin is a fluorescent material, when it absorbs light energy at a wavelength of 488 nm (excitation), it undergoes an electronic state change and instantaneously emits light at 595 nm (emission). The Relative Fluorescence Units (RFU) shown in FIG. 1 indicate the peaks of light emitted at 595 nm resulting from the absorption of Dox at 488 nm. The results depicted in FIG. 1 represent the average of 5 experiments and show that Fms-Dox (gray columns) is orally absorbed.

### Example 3

### Fms-Dox is Potent Against F10.9 Melanoma Cell Line in vitro

The efficacy of native Dox and FMS-Dox on the inhibition of proliferation of cancer cells was tested in vitro with F10.9 melanoma cell line. On day (−1) cells, were seeded in a 24-well plate (5×10³ cells/ml/well). On day (0), cells were incubated with Dox and FMS-Dox at logarithmic dilutions from 100 μM to 0.001 μM. Survival of cells at 96 hours was determined, using the crystal violet staining method. Optical density (OD) was measured at 620 nm in an ELISA reader. The OD reading was correlated to % viable cells. When the cells were untreated the absorbance was at a maximum. IC₅₀ (the concentration at which cell growth is inhibited by 50%) was determined. The results in FIG. 2 show IC₅₀ values of 0.03 μM and 0.09 μM for native Dox and FMS-Dox, respectively. Thus, Fms-Dox shows efficacy comparable to the native doxorubicin in inhibiting proliferation of F10.9 melanoma cell line in vitro. The Fms-Dox activity is due to its cleavage in the cell medium and full recovery of the native doxorubicin. The was confirmed by HPLC analysis whereby the compound found in the growing cell medium after 96 hr corresponded to native doxorubicin.

### Example 4

### In vivo Activity of Fms-Dox

For evaluation of the antineoplastic activity of Fms-Dox in vivo, using the F10.9 melanoma cell animal model, the following protocol is used. Female C57BL/6 mice (one control group and 6 treatment groups of 10 mice each) are injected intravenously (i.v.) on day (0) with 5×106 B16F10.9 melanoma tumor cells/mouse. Mice are divided into 7 groups: (1) control untreated mice (PBS); (2) mice treated with native Dox (3 mg/kg/day) administered I.P. starting on day (1) for 5 consecutive days; (3) mice treated with Fms-Dox (4.5 mg/kg/day) administered I.P. starting on day (1) for 5 consecutive days; (4) mice treated with Fms-Dox (45 mg/kg/day) administered I.P. starting on day (1) for 5 consecutive days; (5) mice treated with native Dox (3 mg/kg/day) administered orally starting on day (1) for 5 consecutive days; (6) mice treated with Fms-Dox (4.5 mg/kg/day) administered orally starting on day (1) for 5 consecutive days; and (7) mice treated with Fms-Dox (45 mg/kg/day) administered orally starting on day (1) for 5 consecutive days. After 4-6 weeks, depending on long-term survivors, the antitumor effect as determined by increase of survival time and reduction of the metastatic index is evaluated by standard methods.

### Example 5

### Preparation of Fmoc-Met-enkephalin and Fmoc-Leu-enkephalin

Met-enkephalin was prepared by conventional solid phase peptide synthesis, with ABIMED, AMS-422 automated solid phase multiple peptide synthesizer (Langenfeld, Germany). In each reaction vessel, 12.5 μmol of Wang resin was used which contained the first, i.e. methionine, covalently bound, corresponding amino acid (typical polymer loadings of 0.3-0.7 mmols/g resin were employed). The side chain protecting group tert-butyl-ether (t-But) was used for tyrosine. Coupling was achieved, as a rule, using corresponding Fmoc-amino acids (50 μmol, 4 eqv.) and PyBop (benzotriazole-1-oxy-tris-pyrrolidino-phosphonium-hexafluoro-phosphate) (50 μmol, 4 eqv.) as a reagent, and 100 μmol of N-methyl-morpholine (NMM), all dissolved in DMF, typically for 20-45 min at room temperature. The N-terminal Fmoc protecting group was not removed and cleavage of the peptide from the polymer was achieved by reacting the resin with trifluoroacetic acid (TFA)/H₂O/triethylsilane (90:5:5) for 1.5 hours at room temperature. The peptide was purified by using a prepacked LiChroCart RP-18 column (250×10 mm, 7 μm bead size), employing a binary gradient formed from 0.1% TFA in H₂O (solution A) and 0.1% TFA with 25% H₂O in acetonitrile (Solution B). The column effluents were monitored by UV absorbance at 220 nm. Electrospray mass spectrometry confirmed the expected molecular weight: m/z 794.

Leu-enkephalin is prepared in the same way and is tested for its antinociceptive (analgesic) properties as described below for Fmoc-Met-enkephalin.

### Example 6

### Fmoc-Met-enkephalin has Analgesic Activity

The purpose of the following experiment was to evaluate the potential antinociceptive (analgesic) properties of Fmoc-Met-enkephalin as compared to its parent native Met-enkephalin peptide, that is known to have an analgesic effect but is not very effective when administered intracerebroventricularly (ICV), probably due to its very rapid destruction by peptidases.

The method used in the experiments herein for evaluating the antinociceptive properties of Fmoc-Met-enkephalin is a standard method for evaluating compounds having analgesic activity such as non-steroidal anti-inflammatory agents, opioids, and other analgesics, and consists in administering acetylcholine I.P. to mice, treating the mice with the test compound and evaluating the inhibition of abdominal constriction induced by the acetylcholine.

Thus, 120 healthy CD-1, Swiss derived, albino male mice (obtained from Charles River Breeding Labs, Wilmington, Mass.) were weighed, examined for health and equally distributed into one of the 12 test groups shown in Table 1.

TABLE 1

| | | | | Type of |
|---|---|---|---|---|
| Group # | Test Substance | Dose Level | | Administration |
| 1 | Vehicle (PBS buffer) | — | | SC |
| 2 | Fmoc-Ala | 25 | mg/kg | SC |
| 3 | Fmoc-Met-enkephalin | 50 | mg/kg | SC |
| 4 | Fmoc-Met-enkephalin | 25 | mg/kg | SC |
| 5 | Fmoc-Met-enkephalin | 12.5 | mg/kg | SC |
| 6 | Fmoc-Met-enkephalin | 6.25 | mg/kg | SC |
| 7 | Met-enkephalin | 50 | mg/kg | SC |
| 8 | Fmoc-Ala | 25 | mg/kg | PO |
| 9 | Fmoc-Met-enkephalin | 50 | mg/kg | PO |
| 10 | Fmoc-Met-enkephalin | 25 | mg/kg | PO |
| 11 | Fmoc-Met-enkephalin | 12.5 | mg/kg | PO |
| 12 | Met-enkephalin | 50 | mg/kg | PO |

SC—subcutaneous, PO—per os (orally).

SC administration was carried out with a standard syrine and needle and given in a volume of 10 ml per kg. Oral administration was performed using a standard curved oval gavage needle and given in a volume of 10 ml per kg. Thirty minutes after administration of the appropriate treatment, acetylcholine (5.5 mg/kg) was injected I.P. The mice were then placed in large plastic boxes and observed for the occurrence of a single abdominal constriction (defined as a wave of construction and elongation passing caudally along the abdominal wall, accompanied by a twisting of the trunk and followed by extension of the hind limbs). Antinociceptive activity was indicated by a statistically significant decrease in the number of mice showing abdominal constriction induced by acetylcholine. Acetylcholine-induced abdominal constriction was repeated again at approximately 120 minutes after test compound administration.

The mice were observed for signs of gross toxicity and/or behavioral changes during the experimental period. Observations included gross evaluation of the skin and fur, eyes and mucous membranes, respiratory, circulatory, autonomic and central nervous system, somatomotor activity and behavioral patterns. Particular attention was directed to observation of tremors, convulsions, salivation, diarrhea, sleep and coma. A non-parametric analysis of quantal data (Fisher exact test) was used to determine statistical significance of results. A difference from the placebo control with p<0.05 was considered to be statistically significant.

Fmoc-Met-enkephalin was tested against acetylcholine-induced abdominal constriction in mice. Antinociceptive activity was evaluated at 30 and 120 minutes after subcutaneous or oral administration. The results are shown in Table 2.

TABLE 2
Effect of SC and PO administered compounds on acetylcholine-
induced abdominal constriction.

| | | # mice | # mice |
|---|---|---|---|
| | | abdominal | abdominal |
| | | construction | construction |
| Group | | (% inhibition) | (% inhibition) |
| No. | Treatment | 30 minutes | 120 minutes |
| 1 | Vehicle (PBS) SC | 10/10 | 8/8 |
| 2 | Fmoc-Ala 25 mg/kg SC | 10/10 | 10/10 |
| 3 | Fmoc-Met-Enk 50 mg/kg SC | 5/10(50)* | 9/10(10) |
| 4 | Fmoc-Met-Enk 25 mg/kg SC | 6/10(40)* | 6/10(40)* |
| 5 | Fmoc-Met-Enk | 7/10(30) | 8/10(20) |
| | 12.5 mg/kg SC | | |
| 6 | Fmoc-Met-Enk | 9/10(10) | 8/10(20) |
| | 6.25 mg/kg SC | | |
| 7 | Met-Enk 50 mg/kg SC | 8/10(20) | 10/10 |
| 8 | Fmoc-Ala 25 mg/kg PO | 10/10 | 8/10(20) |
| 9 | Fmoc-Met-Enk 50 mg/kg PO | 8/10(20) | 6/10(40) |
| 10 | Fmoc-Met-Enk 25 mg/kg PO | 6/10(40) | 10/10 |
| 11 | Fmoc-Met-Enk | 6/10(40)* | 10/10 |
| | 12.5 mg/kg PO | | |
| 12 | Met-Enk 50 mg/kg PO | 9/10(10) | 9/10(10) |

*Statistically significant difference from vehicle or Fmoc control p < 0.05. Fisher's Exact test.

Exact test.

Fmoc-Met-enkephalin was found to produce a statistically significant inhibition of acetylcholine-induced abdominal constriction at 50 and 25 mg/kg SC and 25 and 12.5 mg/kg PO. Fmoc-Ala (25 mg/kg) and Met-enkephalin (50 mg/kg) were not active after SC or PO administration. When evaluated at 120 minutes after drug administration, Fmoc-Met-enkephalin was active at 25 mg/kg SC. Fmoc-Met-enkephalin did not show statistically significant activity at any dose when compared to Fmoc-Ala after oral administration at 120 minutes. The 50-mg/kg dose produced an inhibition of acetyl-choline-induced abdominal constriction that was not statistically significant when compared to Fmoc-Ala but would be statistically significant if compared to 120 minute subcutaneous controls.

Fmoc-Met-enkephalin shows significant antinociceptive activity in the mouse, while neither Met-enkephalin nor Fmoc-Ala is active at comparable doses. The effect is similar in character to that observed after ICV administration of Met-enkephalin and indicates that the Fmoc-Met-enkephalin complex improves or facilitates access of the active agent into the brain. This study also indicates that the Fmoc complex protects Met-enkephalin from destruction in the gastrointestinal tract and in some way facilitates transport through the intestinal mucosa. Oral effectiveness of the highest dose may have been decreased by local effects delaying gastric emptying. The mice had access to food during the experiment and this may also have adversely affected the speed on onset of absorption. In this study, mice did not show signs of CNS depression or sedation ruling this out as a factor in the antagonism of the abdominal constriction due to acetylcholine.

A dose response of Fmoc-Met-enkephalin against acetylcholine-induced writhing was tested in mice with 6.25, 12.5, 25 and 50 mg/kg Fmoc-Met-enkephalin in comparison to the native peptide Met-enkephalin (50 mg/kg) or vehicle (PBS, 10 ml/kg—control). Thirty minutes after SC administration of the appropriate treatment, acetylcholine (5.5 mg/kg) was injected intraperitoneally (10 mice per group). The mice were then placed in large plastic boxes and observed for the occurrence of a single abdominal constriction. The results are shown in FIG. 3. Fmoc-Met-enkephalin was found to produce a statistically significant (p<0.05) inhibition of acetylcholine-induced abdominal constriction at 50 and 25 mg/kg SC. Fmoc-Met-enkephalin showed significant antinociceptive activity in the mouse, while the native peptide was not significantly active. The effect was similar in character to that observed after ICV (direct cerebral injection) administration of the native parent peptide.

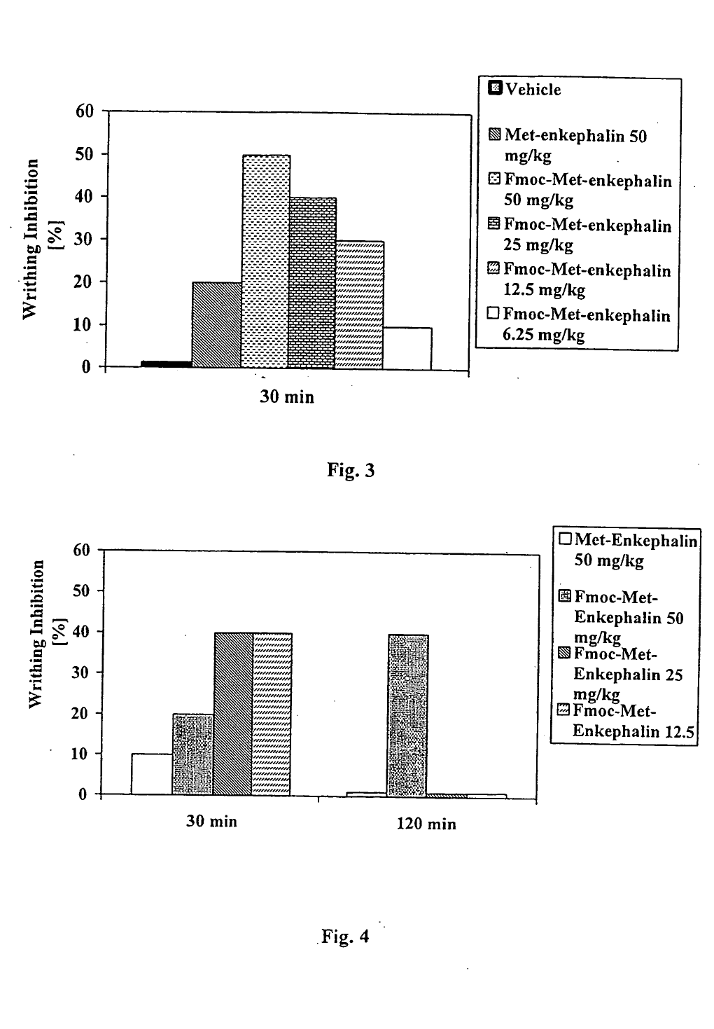

Fmoc-Met-enkephalin was tested for antinociceptive activity after oral administration in 3 different doses—12.5, 25 and 50 mg/kg. Thirty and 120 minutes after administration of the appropriate treatment, acetylcholine (5.5 mg/kg) was injected intraperitoneally (10 mice per group). The mice were then placed in large plastic boxes and observed for the occurrence of a single abdominal constriction. The results are shown in FIG. 4. Fmoc-Met-enkephalin was found to produce a statistically significant inhibition of acetylcholine-induced abdominal constriction at 25 and 12.5 mg/kg after 30 minutes and at 50 mg/kg after 120 minutes, when administered orally. The native peptide is known to be active only after direct injection into the brain. This experiment indicate that the Fmoc moiety protects the native parent peptide from destruction in the gastrointestinal track and in some way facilitates transport through the intestinal mucosa.

### Example 7

### Synthesis of Fms-Leu-Enkephaline and Fms-Met-Enkephalin

Leu-Enkephaline (1 equivalent) dissolved in a 1:1 (v:v) solution of dioxane and water was reacted with 2 equivalents of Fms-OSu at pH 8.5 in the presence of NaHCO₃. The reaction mixture was stirred for 1 hour before another portion of Fms-OSu was added. The pH was again adjusted to 8.5 and the reaction mixture was allowed to stir for 2 additional hours. Analysis was performed by HPLC, employing a binary gradient utilizing 0.1% TFA in water (solution A) and 0.1% TFA in 25% water in acetonitrile (solution B) increasing the fraction of solution B from 10 to 90% from 0 to 40 minutes, at a flow of 0.8 ml/min, and with a UV detection at 220 nm. The crude product was purified by preparative HPLC employing binary gradient: 0-10 min, 10% B; 10-90 min, 10-40% B; 90-100 min, 40-60% B; 100-105 min, 60-100% B, at a flow rate of 12.5 ml/min and with a detection at 220 nm. Mass spectroscopy gave a molecular ion at m/z 875.

Fms-Met-enkephalin is prepared in the same way. Both Fms-Met-enkephalin and Fms-Met-enkephalin are tested for analgesic activity in the same way as described for Fmoc-Met-enkephalin in Example 6 above.

### Example 8

### Synthesis of (Fms)₃-Gentamicin

Gentamicin sulphate (10 mg, 20 μmole) was dissolved in 1.0 ml of 0.5M NaHCO₃ (pH 8.5) and cooled to 0° C. Fms-OSu was added gradually in several aliquots over a period of 2 hours. Overall, 10 molar excesses of Fms-OSu were added over gentamicin. The reaction mixture was then dialyzed against H₂O at 37° C. for three days and finall lyophilized. A dialysis bag with a cut off of 1200±50 daltons was used, which allowed for the diffusion of excess reagents and any residual amounts of native gentamicin or mono- and bis-Fms-Gentamicin.

(Fms)₃-Gentamicin was obtained with >95% purity and an overall yield of 56%. The compound was purified by HPLC with a retention time of 31.4 min. Electrospray mass spectrometry confirmed the expected molecular weight with m/z 1384.2.

In the same way, (Fmoc)₃-Gentamicin and corresponding Fmoc and Fms derivatives of streptomycin and tobramycin can be obtained.

### Example 9

### Antibacterial Potency of (Fms)₃-Gentamicin

A suspension of Escherichia coli (E. coli strain N-4156-W.T, 1% V/V in L.B. medium) is divided into plastic tubes (0.5 ml per tube) and incubated in a shaking water bath at 37° C., in either the absence or the presence of increasing concentrations of gentamicin (0.02-50 μM) and increasing concentrations of (Fms)₃-gentamicin (0.02-50 μM). E. coli replication is evaluated by measuring the absorbance at 600 nm. Incubation is terminated when O.D_{600 nm} in the tubes containing no gentamicin reaches a value of 0.6±0.1. Under our assay conditions, native gentamicin inhibited half-maximally E. coli replication at a concentration of 0.22±0.02 μM (0.1 μg/ml). A Fmoc- or Fms-gentamicin showing an I.C₅₀ value of 2.2±0.2 μM in this assay is considered having 10% the antibacterial potence of native gentamicin.

### Example 10

### Synthesis of of Fmoc-amphotericin B and Fms-amphotericin B

10a. Synthesis of Fmoc-amphotericin B

Amphotericin B (1 equiv) is reacted with Fmoc-OSu (2 equiv) in FMS or DMSO to which 2-3 equivalents of diisopropyl ethylamide (DIPEA) are added to apparent pH 8. The reaction mixture is then stirred for at least 2 hours until analytical HPLC indicates that the reaction is over. The crude material is checked by analytical HPLC (Chromolith C-18 column; 100×4.6 mm) employing a binary gradient formed form solution A. 0.1% TFA in H₂O and solution B: acetonitrile 75% in H₂O in. The gradient is: t=0 7 min, 30 to 100% B; t=7-9 min, 100% B. UV detection at 405 nm. Retention time (amphotericin): 5.3 min aprox. Purification is carried out by preparative HPLC in the same column using a binary gradient of water (solution A) and acetonitrile (solution B): t=0-10 min, 20% B, t=10-90 min, 20-100% B. Detection at 405 nm.

10b. Synthesis of Fms-amphotericin B

Method a. In this experiment, Fungizone was used as source of amphotericin B, where it is formulated as deoxycholate. Thus, 25 mg Amphotericin B in an aqueous solution (27 μmol, 5 mg/ml) were reacted with FMS-OSu (92 mg, 170 μmol) which was added in two portions, separated by an hour. The reaction mixture was stirred for a total of 3 hours. The pH was kept at 8-8.5 with 1M Na₂CO₃. The crude material was checked by analytical HPLC, isocratic run (0.038M ammonium acetate/acetonitrile, 60/40), 20 minutes, and detection at 405 nm, column C18. Two peaks were observed in the HPLC, at approximately 10 and 11 minutes. Purification of the Fms-amphotericin B was achieved with preparative HPLC, gradient: 0.038M ammonium acetate/acetonitrile (80/20 to 60/40), detection at 405 nm. Yield: about 7 mg MS of m/z 1224.

Method b. 10 mg Amphotericin B (0.011 mmol) were dissolved in a 2:1 mixture of DMF/DMSO. Next were added 23 mg of Fms-OSu (0.055 mmol) and 11.5 μL of DIPEA (0.066 mmol). The mixture was stirred for at least two hours. The crude was checked by analytical HPLC (Chromolith C-18 column; 100×4.6 mm) with a binary gradient: solution A: H₂O 0.1% TFA; solution B: acetonitrile 75% in H₂O. t=0 to 7 min, 30 to 100% B; t=7 to 9 min, 100% B. Detection at 405 nm. Rt (amphotericin)—5.3 min aprox., Tr (Fms-amphotericin)—5.9 min aprox. Purification of Fms-amphotericin B was achieved by preparative HPLC employing a binary gradient: A—H₂O; B—acetonitrile. t=0 to 10 min, 20% B; t=10 to 90 min, 20 to 100% B. Detection at 405 nm. Fms-amphotericin B was obtained in 78% yield.

### Example 11

### Evaluation of Oral Absorption of Fmoc- and Fms-amphotericin B

In an assay for evaluating the oral absorption of Fms-amphotericin B and/or Fmoc-amphotericin B after entering into the blood circulation, blood of Wistar rats and CD1 mice are collected following oral administration of the compound. As a control, mice are treated orally or IP with native antifungal amphotericin B. Blood samples from the animals are dripped on round 1 mm filter papers that are placed on an Agar-RPMI plates confluent with Candida albicans. The plates are incubated for 48 hr at 30° C. Amphotericin B is known to be a very potent anti-Candida drug and, usually, 20 IU of native Amphotericin B give a clear zone diameter of 1.5 cm. The blood samples obtained from animals treated with the Fmoc or Fms derivative should also give a clear zone.

### Example 12

### Synthesis of Fmoc-D-Lys⁶-GnRH and Fms-D-Lys⁶-GnRH

The parent GnRH analog, D-Lys⁶-GnRH, was synthesized by conventional solid phase peptide synthesis withn ABIMED AMS-22 automated solid phase synthesizer (Langenfeld, Germany) with Rinkamide resin (25 μmol scale) as a polymeric support, following the company's protocol for Fmoc strategy.

12a. Synthesis of Fmoc-D-Lys⁶-GnRH

D-Lys⁶-GnRH (1 equivalent) was dissolved in DMF and reacted with Fmoc-OSu (2equivalents) in the presence of 3 equivalents of DIPEA. The reaction mixture was stirred for 3 hours and was then analyzed by analytical HPLC, employing a binary gradient of 0.1% TFA in water (solution A) and 0.1% TFA in 25% water in acetonitrile (solution B): 0-30 min, 10-100% B; 30-40 min, 100% B, at a flow rate of 0.8 ml/min. Detection at 220 nm. The Fmoc-D-Lys⁶-GnRH with a retention time of 30.3 minutes aprox.showed a molecular ion of 1475 m/z in its MS spectrum.

The product was purified by preparative HPLC, employing the binary gradients above: 0-15 min, 90% A, 10% B; 15-95 min, 10-100% B. Flow: 12.5 ml/min. Detection at 220 nm. Under this gradient the product eluted after 70 min.

12b. Synthesis of Fms-D-Lys⁶-GnRH

D-Lys⁶-GnRH (1 equivalent) was dissolved in 1:1 mixture of DMF and water and reacted with Fms-OSu (2equivalents) at pH 8.5 in the presence of 1N NaHCO₃. The reaction mixture was stirred for 1 hour before another portion of Fms-OSu was added and the pH readjusted to 8.5. The mixture was allowed to stir for 2 additional hours and was then analyzed by analytical HPLC, employing a binary gradient of 0.1% TFA in water (solution A) and 0.1% TFA in 25% water in acetonitrile (solution B): 0-30 min, 10-100% B; 30-40 min, 100% B, at a flow rate of 0.8 ml/min. Mass Spectral analysis gave a molecular ion of 1555 m/z. The product was purified by preparative HPLC, employing the binary gradient as above: 0-15 min, 10% B; 15-95 min, 10-100% B. Flow: 12.5 ml/min. Detection at 220 nm.

### Example 13

### Evaluation of the Activity of Fmoc- and Fms-D-Lys⁶-GnRH

In order to evaluate whether Fmoc-D-Lys⁶-GnRH or Fms-D-Lys⁶-GnRH are orally absorbed, the effect of these GnRH derivatives on the level of luteinizing hormone (LH) in circulating blood of rates was tested. The experiment was carried out with 55 female Wistar 8-week old rates, equally distributed into one of the eleven test groups as detailed in the following Table 3:

TABLE 3

| | | Dose Level | Type of |
|---|---|---|---|
| | Test Substance | μg/rat | Administration |
| 1 | D-Lys⁶-GnRH | 0.5 | IP |
| 2 | Fms-D-Lys⁶-GnRH | 0.6 | IP |
| 3 | Fmoc-D-Lys⁶GnRH | 0.6 | IP |
| 4 | Vehicle (PBS buffer) | | IP |
| 5 | D-Lys⁶-GnRH | 0.5 | PO |
| 6 | D-Lys⁶-GnRH | 50 | PO |
| 7 | Fms-D-Lys⁶-GnRH | 0.6 | PO |
| 8 | Fms-D-Lys⁶-GnRH | 60 | PO |
| 9 | Fmoc-D-Lys⁶-GnRH | 0.6 | PO |
| 10 | Fmoc-D-Lys⁶-GnRH | 60 | PO |
| 11 | Vehicle (PBS) buffer) | | PO |

The compounds were administered either orally (PO) or intraperitoneally (IP) in a molar concentration of 0.4 nmol/rat (equivalent to 0.5 μg/rat of D-Lys⁶-GnRH and to 0.6 μg/rat of Fms- or Fmoc-D-Lys⁶-GnRH), or of 40 nmol/rat (equivalent in 50 μg/rat of D-Lys⁶-GnRH and to 60 μg/rat of Fms- or Fmoc-D-Lys⁶-GnRH), in 0.5 ml PBS. Ninety and 180 minutes after administration of the appropriate treatment, blood samples were collected (1 ml), the serum was separated, and the detection of LH levels was carried out by radioimmunoassay (RIA) as previously described (Rahimipour, S., et Al. J. Med. Chem. 2001, 44, 3645-3652). The results are shown in Table 4.

TABLE 4
Effect of IP or PO administration of GnRH analogs on LH levels in blood

| | | Dose | Type of | LH | |
|---|---|---|---|---|---|
| Group | Test | Level | Admin- | 90 min | |
| # | Substance | μg/rat | istration | (ng/ml) | LH 180 min |
| 1 | D-Lys⁶-GnRH | 0.5 | IP | 35 | 12 |
| 2 | Fms-D-Lys⁶GnRH | 0.6 | IP | 80 | 25 |
| 3 | Fmoc-D-Lys⁶GnRH | 0 6 | IP | 50 | 17 |
| 4 | Vehicle (PBS buffer) | | IP | 0.3 | 0 |
| 5 | D-Lys⁶-GnRH | 0.5 | PO | 0.3 | 0 |
| 6 | D-Lys⁶-GnRH | 50 | PO | 10 | 0 |
| 7 | Fms-D-Lys⁶-GnRH | 0.6 | PO | 0.4 | 0 |
| 8 | Fms-D-Lys⁶-GnRH | 60 | P0 | 80 | 35 |
| 9 | Fmoc-D-Lys⁶-GnRH | 0.6 | PO | 0 | 0 |
| 10 | Fmoc-D-Lys⁶-GnRH | 60 | PO | 0 | 0 |
| 11 | Vehicle (PBS buffer) | | PO | 0 | 0 |

As shown, oral administration of Fms-D-Lys⁶-GnRH causes an elevation of the LH levels in the blood circulation measured 90 and 180 min after administration. In addition, both Fmoc-D-Lys⁶-GnRH and Fms-D-Lys⁶-GnRH are active when administered IP.

### Example 14

### Synthesis of Fms-Leuprolide and Evaluation of its Activity

14a. Synthesis of Leuprolide

Pyro-Glu-WHSYdLLRP was prepared in the laboratory of the inventors by conventional solid phase peptide synthesis with ABIMED AMS-22 automated solid phase synthesizer (Langenfeld, Germany) with 2-chlorotrityl resin (25 μmol scale) as a polymeric support, following the company's protocol for the Fmoc strategy. The protected peptide was removed from the resin following the company's protocol. The protected peptide was reacted with excess ethylamine using PyBOP as coupling agent. The C-terminal amidated protected peptide thus obtained was treated with TFA/Triethylsilane (TES)/water for 2 hours. The deprotected C-terminal aminated peptide, Leuprolide, was purified by HPLC and analyzed structurally ascertained by mass spectra and by amino acid hydrolysis.

14b. Synthesis of Fms-Leuprolide

Leuprolide (1 equiv) was dissolved in dioxane/water 9:1 and the solution was cooled with ice. FMS-OSu (2equiv) and 1M NaHCO₃ (6 equiv) were added to the solution and the mixture was stirred for 3 hours at 0° C. and then overnight at room temperature. The reaction was quenched with acetic acid. The reaction mixture was monitored by analytical HPLC: Column Chromolith C18, 100×4.6 mm; binary gradient: A (water, 0.1% TFA), B (0.1% TFA in 25% water in acetonitrile)—0 to 8 min: 20 to 100% B, 8 to 9 min: 100% B. Flow: 3 ml/min. UV detector: 220 nm. Retention time: Rₜ (FMS-leuprolide) ˜5.8 min. Mass spectrometry: m/z 1511.

The reaction mixture was purified by preparative HPLC on a C4 column, 250 ×25 mm, with a binary gradient: A (water, B (acetonitrile)—0 to 10 min: 10% B; 10 to 70 min: 10 to 80% B, and 70 to 80 min: 100% B. Flow: 10 ml/min. UV detector: 220 nm. Retention time: Rₜ (FMS-leuprolide) ˜43 min. Mass spectrometry: m/z 1511. Yield 30.

Fms-leuprolide is tested for oral absorption as described above for Fmoc-D-Lys⁶-GnRH and Fms-D-Lys⁶-GnRH.

1-34. (canceled)

35: A compound of the formula:
X—Y
wherein
Y is a moiety of an orally nonabsorbed or poorly absorbed drug bearing at least one functional group selected from free amino, hydroxyl, mercapto, phosphate and/or carboxyl, and
X is at least one radical selected from the group consisting of radicals of the formulas (i) to (iv): 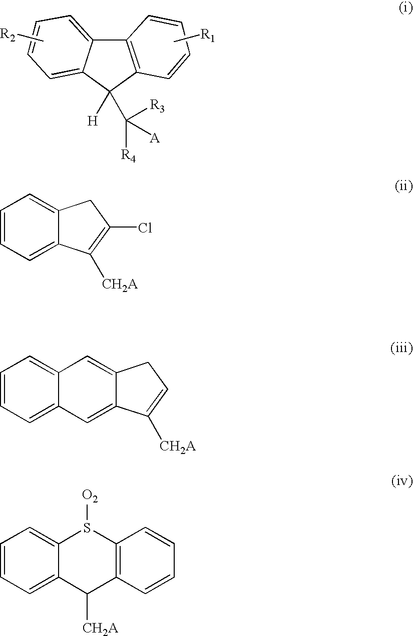
wherein R₁ and R₂, the same or different, are each hydrogen, alkyl, alkoxy, alkoxyalkyl, aryl, alkaryl, aralkyl, halogen, nitro, sulfo (SO₃H), amino, ammonium, carboxyl, PO₃H₂, or OPO₃H₂; R₃ and R₄, the same or different, are each hydrogen, alkyl or aryl; and A is a covalent bond when the radical is linked to a carboxyl, phosphate or mercapto group of the drug Y, or A is OCO— when the radical is linked to an amino or hydroxyl group of the drug Y; and
pharmaceutically acceptable salts thereof.

36: A compound according to claim 35, wherein X is at least one radical (i) wherein R₁ is hydrogen or sulfo and R₂, R₃ and R₄ are hydrogen.

37: A compound according to claim 36, wherein free amino and/or carboxyl groups, and optionally free hydroxyl groups, of the drug moiety Y, are substituted by at least one said radical (i).

38: A compound according to claim 37, wherein one or more amino and/or hydroxyl groups of the moiety Y are substituted by the radical (i) in which R₁ to R₄ are hydrogen and A is OCO— (9-fluorenylmethoxycarbonyl, hereinafter “Fmoc”).

39: A compound according to claim 37, wherein one or more amino and/or hydroxyl groups of the moiety Y are substituted by the radical (i) in which R₁ is sulfo at position 2, R₂, R₃ and R₄ are hydrogen, and A is OCO— (2-sulfo-9-fluorenylmethoxycarbonyl, hereinafter “Fms”).

40: A compound according to claim 37, wherein one or more carboxyl, mercapto or phosphate groups are substituted by the radical (i) in which R₁ to R₄ are hydrogen and A is a covalent bond (9-fluorenylmethyl, hereinafter “Fm”).

41: A compound according to claim 35, wherein one or more amino groups are substituted by the Fmoc or Fms radical and one or more carboxyl groups are substituted by the Fm radical.

42: A compound according to claim 35, wherein one or more hydroxyl groups are substituted by the Fmoc or Fms radical and one or more carboxyl groups are substituted by the Fm radical.

43: A compound according to claim 35, wherein Y is a moiety of an orally nonabsorbed or poorly absorbed drug containing at least one amino-sugar moiety.

44: A compound according to claim 43, wherein said drug containing an amino-sugar moiety is selected from the group consisting of an anthracycline antibiotic, an antibacterial aminoglycoside antibiotic or an antifungal polyene antibiotic.

45: A compound according to claim 44, wherein said anthracycline antibiotic is daunorubicin or doxorubicin.

46: A compound according to claim 45, selected from the group consisting of Fmoc-daunorubicin, Fms-daunorubicin, Fmoc-doxorubicin and Fms-doxorubicin.

47: A compound according to claim 43, wherein said antibacterial aminoglycoside antibiotic is selected from the group consisting of streptomycin, tobramycin and gentamicin.

48: A compound according to claim 47 consisting of (Fmoc)₃-gentamicin or (Fms)₃-gentamicin.

49: A compound according to claim 44, wherein said antifungal polyene antibiotic is amphotericin B.

50: A compound according to claim 49, consisting of Fmoc-amphotericin B or Fms-amphotericin B.

51: A compound according to claim 35, wherein Y is a moiety of an orally nonabsorbed or poorly absorbed beta-lactam antibiotic in which the carboxyl group is substituted by a radical Fm.

52: A compound according to claim 51, wherein said beta-lactam antibiotic is ceftazimide or meropenem and said compound is Fm-ceftazimide or Fm-meropenem.

53: A compound according to claim 35, wherein Y is a moiety of an orally nonabsorbed or poorly absorbed peptide.

54: A compound according to claim 53, wherein said peptide belongs to the endorphin class and is Met⁵-enkephalin (SEQ ID NO: 1), or Leu⁵-enkephalin (SEQ ID NO: 2).

55: A compound according to claim 53, wherein said compound is selected from the group consisting of Fmoc-Met⁵-enkephalin (SEQ ID NO: 3), Fms-Met⁵-enkephalin (SEQ ID NO: 3), Fmoc-Leu⁵-enkephalin (SEQ ID NO: 4), and Fms-Leu⁵-enkephalin (SEQ ID NO: 4).

56: A compound according to claim 53, wherein said peptide is a peptide hormone selected from the group consisting of gonadotropin releasing hormone (GnRH) (SEQ ID NO: 5), an analogue thereof, and octreotide (SEQ ID NO: 15).

57: A compound according to claim 56, wherein said GnRH analogue is selected from the group consisting of leuprolide (SEQ ID NO: 6), nafarelin (SEQ ID NO: 7), goserelin (SEQ ID NO: 8), histrelin (SEQ ID NO: 9) and D-Lys⁶-GnRH D-Lys-GnRH (SEQ ID NO: 10).

58: A compound according to claim 57, of the sequence denoted as SEQ ID NO: 11:
R₅-5-oxo-Pro-His-Trp-Ser-Tyr-R₆-Leu-Arg-Pro-R₇
wherein R₅ is a Fmoc or Fms substitution at a free amino or hydroxyl group of an amino acid residue; R₆ is Gly or a D-amino acid residue of a natural or nonnatural amino acid, and R₇ is Gly-NH₂, NHCH₂CH₃ or NHNHCONH₂.

59: A compound according to claim 58 wherein R₆ is a D-Leu or D-Lys, or a residue of a non-natural amino acid selected from the group consisting of D-Nal [D-3(2-naphthyl)-alanine], D-Ser(t-Bu) and D-His(N^{τ-}PhCH₂).

60: A compound according to claim 58, consisting of Fms-leuprolide, in which the free hydroxyl group of the tyrosine at position 5 is substituted by Fms, of the SEQ ID NO: 12:

| | |
|---|---|
| 5-oxo-Pro-His-Trp-Ser-(Fms)Tyr-D-Leu- | SEQ ID NO: 12 |
| Leu-Arg-Pro-NHCH₂CH₃ | |

61: A compound according to claim 58 consisting of:

| | |
|---|---|
| 5-oxo-Pro-His Trp-Ser-Tyr-(Fmoc)D- | SEQ ID NO: 13 |
| Lys-Leu-Arg-Pro-Gly-NH₂ | |
| or | |
| 5-oxo-Pro-His-Trp-Ser-Tyr-(Fms)D-Lys- | SEQ ID NO: 14 |
| Leu-Arg-Pro-Gly-NH₂. | |

62: A compound according to claim 56, wherein said peptide hormone is octreotide (SEQ ID NO: 15) and said compound is Fmoc-octreotide or Fms-octreotide, wherein the Lys residue at position 5 is substituted by Fmoc or Fms (SEQ ID NO: 16).

63: A compound according to claim 53, wherein said orally nonabsorbed or poorly absorbed peptide is eptifibatide and said compound is selected from the group consisting of Fmoc-eptifibatide and Fms-eptifibatide, wherein the free amino group of the Lys residue is substituted by Fmoc or Fms.

64: A pharmaceutical composition for oral administration comprising a compound according to claim 35 or a pharmaceutically acceptable salt thereof, and a pharmaceutically acceptable carrier.

65: A method for oral treatment of a disease or disorder that can be treated with an orally nonabsorbed or poorly absorbed drug Y, which comprises administering to an individual in need thereof a suitable amount of a compound X—Y according to claim 35.

66: A method for converting an orally nonabsorbed or poorly absorbed drug Y to an orally absorbed drug suitable for oral delivery, which comprises attaching to at least one free amino, hydroxy, mercapto, phosphate and/or carboxyl group of said orally nonabsorbed or poorly absorbed drug at least one radical selected from the group consisting of the radicals of the formulas (i) to (iv): 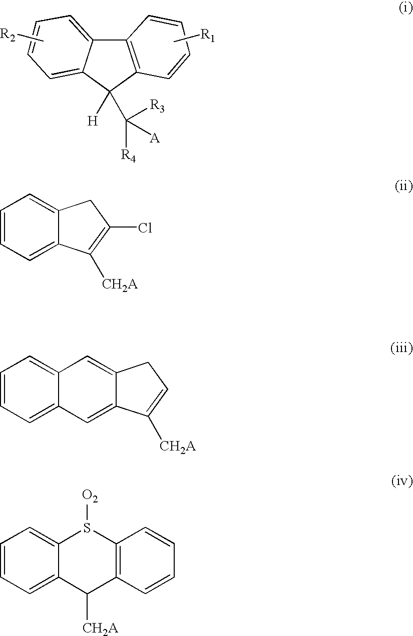
wherein R₁ and R₂, the same or different, are each hydrogen, alkyl, alkoxy, alkoxyalkyl, aryl, alkaryl, aralkyl, halogen, nitro, sulfo (SO₃H), amino, ammonium, carboxyl, PO₃H₂, or OPO₃H₂; R₃ and R₄, the same or different, are each hydrogen, alkyl or aryl; and A is a covalent bond when the radical is linked to a carboxyl, phosphate or mercapto group of the drug Y, or A is OCO— when the radical is linked to an amino or hydroxyl group of the drug Y.

67: A Fmoc- or Fms-derivative of a compound selected from the group consisting of Met-enkephalin, Leu-enkephalin, doxorubicin, gentamicin, amphotericin B, leuprolide and D-Lys⁶-GnRH.

68: A Fmoc- or Fms-derivative according to claim 67 selected from the group consisting of Fmoc-Met-enkephalin, Fms-Met-enkephalin, Fmoc-Leu-enkephalin, Fms-Leu-enkephalin, Fms-amphotericin B, Fmoc-amphotericin B, (Fms)₃-gentamicin, Fms-doxorubicin, Fms-leuprolide, Fms-D-Lys⁶-GnRH and Fmoc-D-Lys⁶-GnRH.
